(19) United States
(12) Patent Application Publication
(10) Pub. No.: US 2004/0253281 A1
(43) Pub. Date: Dec. 16, 2004

(54) Therapeutic markings applied to tissue

(75) Inventors: Steve A. Herweck, Nashua, NH (US); Paul Martakos, Pelham, NH (US); Roger Labrecque, Londonderry, NH (US); Geoffrey Moodie, Nashua, NH (US)

Correspondence Address: LAHIVE & COCKFIELD, LLP., 28 STATE STREET, BOSTON, MA, 02109, US

(73) Assignee: ATRIUM MEDICAL CORP., Hudson, NH

(21) Appl. No.: 10/461,146

(22) Filed: Jun. 12, 2003

Related U.S. Application Data
(63) Continuation-in-part of application No. 10/461,145, filed on Jun. 12, 2003.

(51) Int. Cl. A61K 9/00

(52) U.S. Cl. 424/401

(57) ABSTRACT

A medical ink is loaded with a number of therapeutic agents. The ink is then applied directly to the tissue of a patient, either internally or externally, resulting in a therapeutic ink marking. The therapeutic ink marking can include surface activation of an immobilizing medication, controlled medication release, and/or the ability to use dyes or pigments to delineate different active ingredients by location and dosage. The active medicinal compounds can be placed on selective areas of the tissue as applied in the marking. The marking can provide a detectable and dosemetric controllable delivery to a specific targeted and localized location to provide the maximum therapeutic benefit. The medicated ink may be applied to a number of different methods. Dimensions of the markings can further serve to control and identify to the user the dosage amount of the medical agent applied to the tissue. Multiple types of medical agents with multiple application methods can be used. The medicated ink can be dyed, pigmented, or used as a colorless vehicle, and can be formulated to incorporate either immobilized or exuding active agents onto the tissue.

### RELATED APPLICATION

This application is a continuation-in-part of application Ser. No. ______, entitled “Medicated Ink”, having the Attorney Docket Number ATA-531, filed on the same day as the present application, and which is expressly and entirely incorporated herein by reference.

### FIELD OF THE INVENTION

The present invention relates to an active ink, and more specifically to an ink including an active agent. The ink is applied directly to the tissue of a patient, is detectable, and includes at least one medication, drug, and/or therapeutic agent applied to the patient for therapeutic purposes.

### BACKGROUND OF THE INVENTION

Application of a therapeutic and/or medical agent to the tissue of a patient is known in the art. In some instances, the application occurs through the coating of a medical device with an application of a medical agent for delivering medication to a patient upon usage of the medical device. For example, medical devices, such as balloons or stents, can be coated with one or more agents for controlling restenosis or smooth muscle cell hyperplasia in the human coronary arteries. The balloon or stent can have a drug eluting coating applied to one or more surfaces thereof. With this method, the drug is impregnated or made part of the coating that is applied only to the surface of the medical device structure. Known coating methods provide drug release from a bonded polymeric material or coating that surrounds one or more surfaces of the balloon or stent that generally provide a fixed rate of release of one or more medications.

Alternative to medicated devices, there are often instances where it is desirable to have a drug or agent applied directly to the tissue of a patient. In some instances, there is no need or ability to use a medical device implanted on or in the patient that includes a medicated coating for application to the tissue of the patient. For example, application directly to the skin of a patient can be done without use of a medical device because of easy access to the skin. Alternatively, some applications of medication directly to tissue during surgery may be necessary but without the option of being able to leave an implant within the patient to dispense the medication. If such an implant remains within a patient a subsequent surgery may be required to remove the implant. In other instances it may be desirable to quickly apply medication to specific locations on a patient with specificity. For example, in preparation for a surgical incision, an application of antibiotic to the specific incision location could prevent infection of the incision.

However, many medications or other therapeutic agents that are applied to the tissue of a patient are either undetectable or not differentiable. Application of a clear medication or agent can be easily missed upon subsequent inspection. In addition, medications or agents that evaporate quickly, or penetrate the tissue quickly can also be missed upon inspection after application. Furthermore, most medications or agents are either clear or white in color, thus differentiating one medication or agent from another is nearly impossible. In instances where medications are colors other than clear or white, there is remains no mechanism to clearly distinguish or identify the medication once it is applied to the tissue.

### SUMMARY OF THE INVENTION

It is therefore desirable to mark or print a marking having therapeutic or diagnostic properties directly onto the tissue of a patient. This marking technology provides a detectable application means at the time of application by a clinical user, and further provides a therapeutic and/or diagnostic benefit to a patient.

In accordance with one embodiment of the present invention, a therapeutic ink for forming a therapeutic marking on tissue of a patient is provided. The therapeutic ink includes a component providing an indication of location of the ink when applied to the tissue. At least one therapeutic agent component is also provided for creating a therapeutic effect when the therapeutic ink is applied to the tissue.

In accordance with aspects of the present invention, the marking is applied with a marker. A dosage of the medical agent in the therapeutic ink can be determined by inspection of the marking. A dosage of the at least one therapeutic agent in the therapeutic ink can be controlled by detectable dimensions of the marking on the tissue. A dosage of the at least one therapeutic agent in the therapeutic ink is determinable by visual detection of the marking. At least one therapeutic ink can include two or more therapeutic inks forming the marking, wherein the marking includes a first color and a second color that differs from the first color.

In accordance with further aspects of the present invention, the marking includes more than one type of therapeutic agent. The therapeutic agent can include a diagnostic medical agent. The marking can be permanently applied to the tissue. The marking can be temporarily applied to the tissue. The therapeutic agent can include an antioxidant agent, an antihypertensive agent, an anti-inflammatory agent, a growth factor antagonist, an antiplatelet agent, an anticoagulant agent, a thrombolytic agent, drugs to alter lipid metabolism, an ACE inhibitor, at least one of an antiproliferative and an antineoplastic, a tissue growth stimulant, a chemical donor of at least one of Nitric oxide and Super Oxygenated O2, promotion of hollow organ occlusion or thrombosis agents, a functional protein and factor delivery agent, a second messenger targeting agent, an angiogenic agent, an anti-angiogenic agent, an inhibition of protein synthesis agent, an anti-infective agent, a gene delivery agent, at least one of a tissue perfusion enhancer and a tissue absorption enhancer, a nitric oxide donative derivative, drug carrying nano-particles, drug carrying micro-spheres, at least one of an imaging agent and a contrast agent, an anesthetic agent, a descaling agent, and or a cheomotherapeutic agent.

In accordance with further aspects of the present invention, the therapeutic agent can be suitable for release into the body of the patient. The therapeutic agent can be suitable for release into the tissue of the patient. Further, the therapeutic agent can include an immobilized drug. The therapeutic agent can exude from the marking. The therapeutic agent can also be enclosed in liposomes.

In accordance with further aspects of the present invention, the ink marking can be visible to the human eye without visual aid. The ink marking can be visible to the human eye with use of a visual aid.

In accordance with further aspects of the present invention, the manner in which the ink marking is applied can include at least one of a color code format, a shape format, a symbol format, and a size format to convey information. In addition, the manner in which the ink marking is applied can include application of the ink marking to indicate at least one of drug type, drug brand, drug dosage, dimensions, sizing, placement, orientation, and trimming guidelines.

In accordance with another embodiment of the present invention, a method of applying a detectable therapeutic information conveying marking to tissue of a patient includes providing an applicator holding detectable therapeutic ink. A first marking of the detectable therapeutic ink is applied to the tissue to apply a specific dosage of at least one therapeutic agent, wherein the dosage is controlled by the quantity of the first marking.

In accordance with aspects of the present invention, the tissue can be pre-treated prior to applying the first marking. A second marking can be applied that is detectably different from the first marking. Applying the first marking can include applying multiple therapeutic agents to the tissue.

In accordance with further aspects of the present invention, at least the first marking can be applied to the tissue by a marker pen, a stamp device, using thermal transfer or gas vapor deposition, and/or in the form of a tattoo.

In accordance with further aspects of the present invention, the first marking includes a first color. A second marking can be formed of a second color that is visually differentiable from the first color. The therapeutic ink can be suitable for release into the body of the patient. The therapeutic ink can be suitable for release into the tissue of the patient.

In accordance with another embodiment of the present invention, a method of applying a therapeutic agent to tissue of a patient includes determining a length of detectable information conveying marking to be applied to the tissue according to the amount of medical agent desired and applying the determined length of marking to the tissue.

### BRIEF DESCRIPTION OF THE DRAWINGS

The invention will be more fully understood from the following detailed description taken in conjunction with the accompanying drawings, in which:

FIGS. 1A and 1B are diagrammatic illustrations of a marking as applied to a tissue location on a patient, in accordance with aspects of the present invention;

FIGS. 1C and 1D are diagrammatic illustrations of a marking as applied to a tissue location on a patient subsequent to application of a preparatory substance or coating, in accordance with aspects of the present invention;

FIGS. 2A, 2B, and 2C are diagrammatic illustrations of markings applied in different configurations or patterns, in accordance with aspects of the present invention;

FIG. 3 is a diagrammatic illustration of a marking applied around a target area for a surgical incision, in accordance with aspects of the present invention;

FIG. 4 is a diagrammatic illustration of a marking applied around pre-existing wound, in accordance with aspects of the present invention;

FIG. 5 is a diagrammatic illustration of a marking applied as a stamp or decal, in accordance with aspects of the present invention;

FIGS. 6A, 6B, and 6C are diagrammatic illustrations if ink markings applied in different colors, in accordance with aspects of the present invention;

FIG. 7A is an illustration of a marker applying a marking to the tissue of a patient, in accordance with aspects of the present invention;

FIG. 7B is an illustration of a stamp and ink pad device for applying a marking to the tissue of a patient, in accordance with aspects of the present invention;

FIG. 7C is an illustration of a bar code symbol version of a marking, in accordance with aspects of the present invention; and

FIG. 8 is a flow chart illustrating one example method of applying a marking to the tissue of a patient, in accordance with aspects of the present invention.

### DETAILED DESCRIPTION

An illustrative embodiment of the present invention generally relates to improving the dosing and flexibility of applying different medications, drugs, therapeutic and/or other agents directly to the tissue of a patient. The present invention provides a clinical user with the opportunity to apply and confirm visually, electronically, or by other detection means, the type and/or dosage of an agent applied to a patient via an agent ink. By use of an application device, the user can actually apply and control the amount of ink, and thus agent, marked on to the patient. Detectable marked dimensions and/or color of a marking serves to identify and help control the prescribed dosage amount of the agent when applied to the patient by the clinical user. The markings can relay a variety of information, such as dimensions, drug information, other characteristics, pattern guidelines, and other usage instructions, if desired.

The markings are identifiable or detectable. What is meant by identifiable and detectable is that the markings are not necessarily visible to the un-aided eye, and the information stored within the markings is not necessarily discernable with the un-aided eye. In accordance with some embodiments of the present invention, the markings are identifiable or detectable, including being visually based, such as with specific colors, symbols, patterns, and the like.

Alternatively, in accordance with additional embodiments of the present invention, the markings can be invisible or substantially invisible to the un-aided eye, but can be made visible using any number of devices. For example, the markings can utilize ink that can only be seen if doused in a developing type solution that chemically alters the appearance. The markings can utilize ink that is only visible when, e.g., an infra read or ultra violet, or some other specific wavelength of light is shining on the ink. The markings can also be made visible when a specific temperature of the ink is achieved.

In addition, the markings can be visible, but not readily discernable. For example, the markings can take the form of a bar code, or some other machine vision based code. Such markings are visible, but without electronic or digital translation, the information conveyed by the marking is not readily discernable.

All of the above instances are intended to fall under the general scope of the terms identifiable and detectable as utilized herein. In addition, the markings convey various forms of information useful to the user, as detailed herein below.

Further, the term “markings” as utilized herein is intended to relate to the result of the application of a substance containing a medication, drug, therapeutic agent, adhesive or bonding agent, and/or other agent. The substance can include a form of ink, decal, or the like, that can be detected by a user with and/or without aid of a device after application. The resulting marking results in at least some form of therapeutic or diagnostic benefit to a patient.

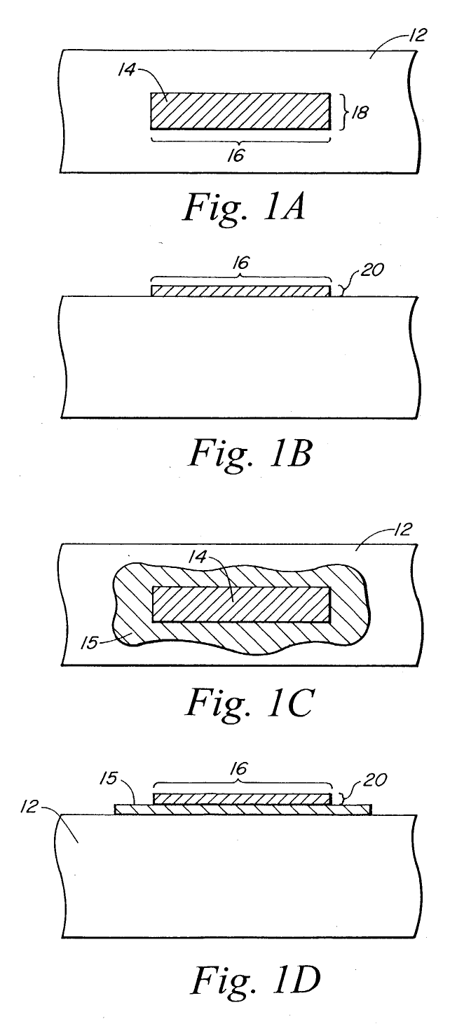

FIGS. 1A through 8, wherein like parts are designated by like reference numerals throughout, illustrate example embodiments of a marking delivery system according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

The teachings of the present invention are applicable both to temporary and permanent markings. A temporarily-placed marking is defined as being a marking that can be removed or will degrade, dissolve, or otherwise dissipate at the conclusion of the therapeutic or diagnostic purpose. A permanently-placed marking, in contrast, stays within the body for an extended period of time, or in perpetuity.

The exemplary embodiments of the present invention further provide a controllable means for identifying a marking, and/or identification of a dosage or release rate of a corresponding agent, at a specific area where the marking denotes on the patient.

FIGS. 1A and 1B illustrate examples wherein a marking is applied to a patient FIGS. 1A and 1B show a marking 14 that has been applied to tissue 12 of a patient. The marking 14 is made by applying an ink that includes an ink carrier component, an agent component, and optionally an adhesive or bonding agent for extended or permanent ink adhesion to the tissue 12. Medication saturation, loading, and dimensions of the marking 14 control the dosage of the agent that is delivered to the patient. The marking 14 can be made visible, or alternately detectable, by accessory device means that applies the marking 14 so that the user can confirm the application and the appropriate dosage applied to the tissue 12. The marking 14 may be visible either to the naked eye, under illumination by selected types of light, or when the user employs accessory detection aids (such as electronic scanner). The dosage of available medication or other agent can also be visibly identified by color or by combination with the dimensions and/or light refraction of the marking 14.

The marking 14 can be applied to the tissue 12 in various shapes and forms. FIGS. 1A and 1B show examples where the marking 14 is applied to the tissue 12. The marking 14 results from an application that includes an agent component. In one embodiment, the amount of agent in the marking 14 corresponds to the dimensional volume of the marking 14. The dimensional volume of marking applied in FIGS. 1A and 1B is equal to the product of length 16, width 18, and height 20 of the marking 14. The amount of agent on the tissue 12 may thus be controlled by varying the dimensions of the marking 14. For example, the amount may be varied by varying the length 16 of the marking 14, the width 18 of the marking 14, or the height 20 (i.e., thickness) of the marking 14. The marking 14 can further be printed in a geometric shape, geometric code, universal bar code, or other format for identification and detection of the agent applied onto the tissue 12. As shown in FIGS. 1C and 1D, the amount of the marking 14 deposited can further be increased by altering the surface of the tissue 12 chemically or otherwise, to alter the ability of the marking to adhere to the tissue 12. For example, the tissue 12 can have a preparatory layer or coating 15 of a substance that improves absorption of the agent in the marking 14 by the tissue 12. The layer or coating 15 can have a number of other results, such as enabling the marking 14 to better adhere to the tissue 12, or to react with the marking 14 upon application of the marking 14 to the tissue 12. The layer or coating 15 can be applied immediately before application of the marking 14, or can be applied at periods of time substantially before application of the marking 14 to have a more extensive effect on the tissue 12.

The surface area of the marking 14 can also affect the rate of delivery of the agent to the patient. In general, a larger surface area results in a higher rate of delivery of the agent than a smaller surface area (given a same concentration of agent). Further, an irregular surface topography, including pores, may either increase or decrease the amount of marking applied to the tissue 12. Hence, a clinical user may wish to consider both the volume and surface area when marking the tissue 12 of a patient.

More specifically, the markings 14 can have different lengths and thicknesses chosen for delivery of the appropriate dosages of the medical agents. In other words, given a uniform number of application layers, increased lengths of markings 14 result in increased dosages of the agents. Therefore, upon quick visual inspection, a user can determine and/or confirm the dosage amount provided. If the thickness is varied, the same length of marking 14 can also result in different dosages.

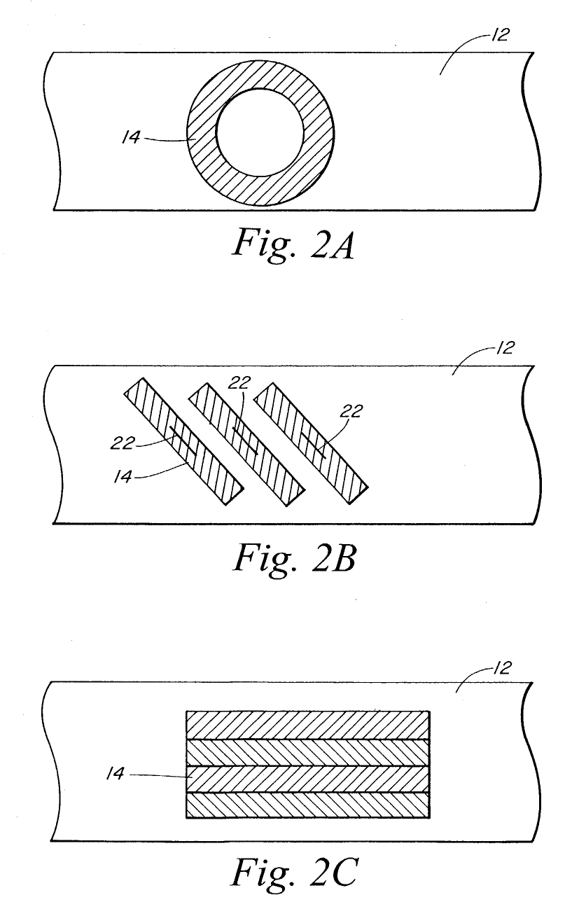

As previously mentioned, the marking 14 can be applied to the tissue 12 in various shapes and forms. FIGS. 2A, 2B, and 2C show examples where the marking 14 is applied to the tissue 12. The marking 14, as applied by a clinical user, can have an essentially infinite number of patterns or designs. FIG. 2A shows the marking 14 in a generally circular shape. The circle can be hollow, as shown, or solid. The circle can be placed on the tissue 12 in a manner that surrounds a wound or other identifiable area on the tissue 12 requiring treatment. The marking can also be placed on top of such an area.

FIG. 2B shows an additional example of the marking 14 in a pattern of angled lines. The lines are disposed over a medical fastening device 22, such as stitches or a staple. The illustration represents the use of the marking 14 as, for example, an anti-inflammatory, anti-microbial, or anti-infective agent place over the medical fastening device 22 to prevent infection. Either before, or after, insertion of the medical fastening device 22, the markings 14 are placed on the tissue 12 in the approximate location of the medical fastening device 22. The agents contained within the marking 14 can be varied for the particular application. Those agents listed relative to FIG. 2B are merely illustrative of example agents or medications.

FIG. 2C shows an example of the marking 14 formed of a series of parallel lines. The parallel lines can be formed of the same ink with the same agent or agents. As shown, the lines are formed of at least two different inks and agents. This illustration shows how multiple inks and agents can form the marking 14 as applied to the tissue 12. With different inks, and more particularly different agents, multiple symptoms or maladies can be treated simultaneously. The different inks and agents can form the markings 14 in whatever combination the clinical user desires, to achieve whatever therapeutic effect attributable to the particular agents being applied in the markings 14.

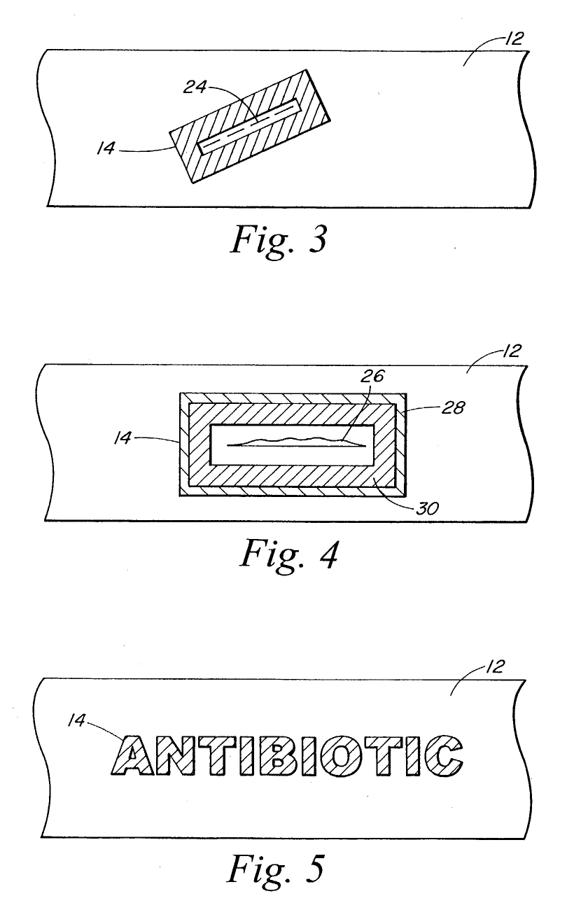

FIG. 3 shows the marking 14 in the general shape of a hollow rectangle. Inside the hollow rectangle shape of the marking 14, a dotted line 24 indicates the location of a future surgical incision. The marking 14 in such an instance can contain a therapeutic agent, such as a sterilization, anti-inflammatory, anti-microbial, and/or anti-infective agent, or some other agent as understood by one of ordinary skill in the art. The marking 14 can both serve to reduce the likelihood of infection of the pending incision, and also serve to help the surgeon visibly identify the location for making the incision. If desired, the marking 14 can be made in such a way as to indicate the desired direction, depth, or other characteristics of the pending incision.

FIG. 4 shows the marking 14 again in the general shape of a hollow rectangle. However, in the example embodiment shown, the marking 14 surrounds an existing incision or wound 26 on the tissue 12 of the patient. If the marking 14 is not present prior to the incision or wound 26 as described in FIG. 3, the marking 14 can be made after the existence of the wound 26 for therapeutic purposes. The marking 14 of FIG. 4 additionally demonstrates an example embodiment wherein the marking 14 is made of two different markings containing two different agents. A first marking 28 and a second marking 30 surround the incision or wound 26. As depicted, the first marking 28 and second marking 30 can be applied in two different arrangements. The first marking 28 can serve as a border that surrounds the second marking 30. In this instance, the agent(s) in the second marking 30 are closer to the incision or wound 26, and thus have a more immediate effect, while the agent(s) in the first marking 28 are more removed from the incision or wound 26, thus having a secondary or delayed effect. Alternatively, the first marking 28 can be applied to the tissue 12 and then the second marking 30 can be applied directly on top of the first marking 28 to form a layered effect. In such an instance, the agent(s) in the first marking 28 are closest to the tissue 12 and the incision or wound 26, thus having a primary effect on the tissue. The agent(s) in the second marking 30 must either wait for the first marking 14 to be absorbed by the tissue 12, or pass through the first marking 28 to reach the tissue 12. Thus, the agent(s) in the second marking 30 have a secondary effect on the tissue 12.

One of ordinary skill in the art will appreciate that there can be any number of layers as shown in FIG. 4 having the same dimensions or different dimensions as applied to the tissue 12. The different layers can contain the same or different agents. For example, to increase the dosage of a particular agent in a specified location on the tissue 12, multiple layers of markings 14 can be made over the specified location. Each layer is an added dosage amount. Alternatively, different agents can exist in each layer. Thus, for example, an agent that improves tissue absorption can form the first layer or first marking 28, and the therapeutic agent can exist in the second layer or second marking 30 applied on top of the first marking 28. Alternatively, two or more components of a therapeutic agent can be applied in separate markings. For example, the first marking 28 can include a first component of a therapeutic agent, while the second marking 30 can include a second component of the therapeutic agent. Once the second marking 30 is applied over the first marking 28, each of the components combines to form the therapeutic agent formed on the tissue 12 for the desired therapeutic effect. In addition, the application of the layers can be staggered. For example, the first marking 28 can be applied including a therapeutic agent that has a therapeutic effect on the tissue 12. After a selected period, the second marking 30 is then applied, resulting in an additional therapeutic effect. Such a process can continue as desired with additional layers of markings.

FIG. 5 shows another example embodiment of the marking 14. In this instance, the marking 14 is in a predetermined form, symbol, or word. As shown, the marking 14 is in the form of the word “antibiotic”, which would indicate that the marking 14 includes at least one antibiotic agent. The marking 14 in this instance can be applied using a stamp in the shape of the word or symbol. Alternatively, the marking 14 can be manufactured as a form of decal that can be applied to the tissue 12 keeping the form, symbol, or word in tact. Thus, a stamp or decal form of the marking 14 can be applied by a first clinical user, and then a second clinical user that may or may not have been present at the application of the marking 14 can easily determine where the marking 14 was placed on the tissue and what agent(s) was incorporated in the marking 14 for application to the tissue 12. One of ordinary skill in the art will appreciate that the form, symbol, word, and the like, can take many different forms and can convey information to the clinical user as desired.

The present invention enables a physician to apply the marking 14 at a desired location on the tissue 12, such as at or around the epidermal exit wound. For example, a user can apply antibiotic, analgesic, or anti-inflammatory medicated ink marks on a specific location where the medicated ink marks will provide the most therapeutic benefit. Further, a user can also apply a medicated ink mark to the specific desired location of dialysis needles, dialysis catheters, orthopedic implant or traction pins, laparoscopic devices, or spinal tap needles with detectable confirmation and/or visual confirmation prior to or during medical device usage.

A combination or mixture of a non-medicated ink or other substance with the ink containing the agent to form a blended ink is another method for controlling the rate of delivery of the agent to the patient. With the addition of the non-medicated ink or substance, the amount and rate of activation and/or release of the agent can be made different for different agents and/or different anatomical locations. A second non-medicated ink can further be applied as the second marking 30 to modulate the activation and/or release of the agent from the first marking 28. In addition, the tissue 12 can be pre-treated with a medicated or non-medicated substance to affect absorption by the tissue.

Numerous modifications to marking shape, including pattern and orientation, will be apparent to those skilled in the art in view of the foregoing description. Accordingly, this description is to be construed merely as illustrative of the inventive concept herein. The description and illustrations should not be construed as limiting the invention.

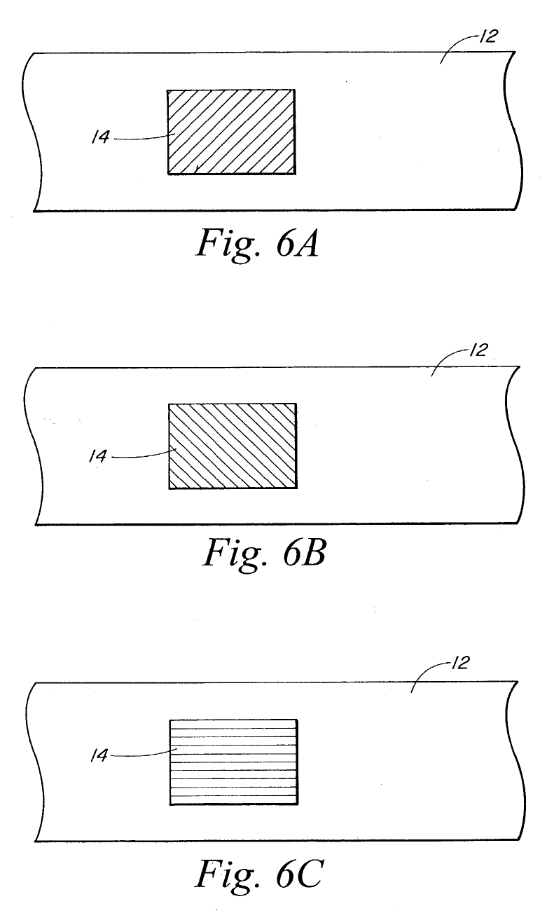

FIGS. 6A, 6B, and 6C illustrate three different embodiments of the marking 14, in the form of three different colors. FIG. 6A shows the marking 14 having a first color. FIG. 6B shows the marking having a second color. FIG. 6C shows the marking having a third color. The marking 14 is shown in the same generally rectangular shape, however, the shape of the marking 14 can vary regardless of the color.

Those skilled in the art will appreciate that a number of different bio-erodable, soluble, or permanent marker inks may be used to create the marking 14. In general, inks are formulated using a pigment to impart color, a resin binder to form the finished ink and carry the pigment, drug exuding medication, or chemical and/or solvent required to enable the binder-pigment mixture to be adhered to the tissue. Suitable pigments include but are not limited to those approved by the USFDA for medical use as listed in Title 21, Sections 73 and 74 of the Code of Federal Regulations (CFR). The following are directly applicable to tissue:

| | |
|---|---|
| Ultramarine blue | FD&C Blue |
| Iron oxide | FD&C Green |
| Titanium oxide | FD&C Red |
| Chromium-cobalt-aluminum oxide | FD&C Yellow |
| Ferric ammonium citrate | D&C Orange |
| Chromium oxide green | D&C Brown |
| Logwood extract | D&C Violet |
| Phthalocyanine green | |

In addition, those of ordinary skill in the art will appreciate that the colors can provide an indication of agent brand name, or an indication of type of agent, associated with the marking 14. For example, if a particular drug has a unique color associated with its identification or trademark, the same color can be replicated in the ink of the marking 14, such that the marking 14 is easily identified as containing that particular drug or agent. Alternatively, the color of the marking 14 can provide an indication of a type of agent found in the marking 14 applied. The use of different colors allows a physician, or other clinical user, to visibly identify the class of medication applied to the tissue 12. The different color schemes for different classification types of medication provide the user with the ability to check and confirm prior to incision or other action, which medication or therapeutic application is incorporated into the ink applied to the tissue 12. Further, the markings 14 can have different dimensional lengths that are chosen for specific dosages for each corresponding agent. The specific color scheme utilized can be standardized by, for example, a national standardizing entity. The color scheme can include solid colors, as shown in FIGS. 6A through 6C, or can include simple patterns of alternating or otherwise differing colors. One of ordinary skill will appreciate the virtually infinite variability of colors, hue, fluorescence, and simple color patterns that can be used to identify particular classes or types of drugs. The colors can identify specific brand names of drugs, or any other desired clinically related attribute, as well.

As previously indicated, medical agents may be added directly to ink formulations to provide the marking 14 with medical properties. Additives and drug carrying nano-particles or microspheres containing medical agents may also be included in the ink formulation to achieve specific rates of medication permeation to local tissue. For example, fast soluble and slow soluble nano-particles or microspheres, organic solvents, and surfactants may be used to achieve a desired ink viscosity to apply the ink onto the tissue 12. The solvent and surfactant are optionally removed in a subsequent process step. Other additives can include plasticizers, bio-erodable components, dye components, adhesives, bonding agents, medication stabilizers, coated and non-coated medical agent nano-particles, or microspheres, designed to improve the ink's flexibility, flow, pigment stability, shelf-life stability, and rate of surface activation and/or release into tissue or body fluid. Inks can also be formulated containing liposomes, with medication enclosed in liposomes, or phospholipid coatings. These inks can be triggered to release active compounds using an internal or external stimulus, such as ultrasound, radiation, magnetic field, or temperature

The following examples illustrate exemplary embodiments of the present invention. A first example involves the use of the present invention in surgery. In particular, a user can make use of a visually detectable marking 14 in orthopedic surgery. In such a surgical procedure, it is often the case that there is a significant amount of blood or other fluids in the vicinity of the procedure. The user can apply the marking 14, and because it can be made with an ink that is highly visually detectable, the user can see where the therapeutic has been applied.

Another application involves laparoscopic surgery, whereby internal tissue visualization and surgical intervention is done solely by video camera and port sealed instrumentation. A laparoscope is placed through a small incision or opening in the patient. The video image is then transmitted back to a video monitor so the surgeon can see where the laparoscope is within the patient. Use of a visually detectable medicated or therapeutic ink by a suitable laparoscopic surgical instrument to form a marking 14 on the tissue 12 internal to the patient facilitates application control and confirmation of therapeutic delivery to the targeted location.

Still another application of the present invention involves the use of radiopaque or otherwise machine detectable ink. In such an instance, the stability or migration of the therapeutic agent applied to a specific targeted location can be confirmed non-invasively by ultrasound, x-ray, MRI, CAT, PET, and the like. For example, the ink can be applied to a specific location during a surgical procedure. Hours or days later, the stability of the ink, or the migration of the ink, can be verified by remote monitoring because of the machine detectable qualities of the ink.

Those skilled in the art will appreciate that a number of different medical agents may be used in the marking 14. For example, anesthetic, anti-infective, lipid lowering, absorption enhancing, anti-oxidant, anti-platelet, cytostatic or cytotoxic medications can be used. In addition, medical agents that promote hollow fluid organ vaso dilation, vaso constriction, occlusion, or thrombosis can be used. The medical agents may include drugs that promote anti-thrombotic activity or can be a clot lysing agent known as a thrombolytic. The medical agents can be kinases or enzymes. The medical agents can be those that promote anti-inflammatory activity or those that promote or stimulate new bone growth. The medical agents can further include agents that promote new cell growth and/or tissue regeneration. The table below (Table #1) summarizes some examples of suitable therapeutic medical agents listed by class.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium phosphate |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, |
| | ranpirnas, troglitzon, valsarten, pemirolast |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |

In addition to or in conjunction with the above table, the medical agent of the present invention can further include an antimicrobial agent. As utilized herein, the term antimicrobial agent shall include antibiotic, antimicrobial, antibacterial, germicidal agents and the like. There may be a combination of antimicrobial agents. In addition, example antibiotics which may be used in conjunction with the present invention include: aminoglycosides, such as gentamicin, kanamycin, neomycin, paromomycin, streptomycin, or tobramycin; ansamycins, such as rifamycin, or rifampin; cephalosporins, such as cephalexin, cephaloridine, cephalotbin, cefazolin, cephapirin, cephradine, or cephaloglycin; chloramphenicols; macrolides, such as erythromycin, tylosin, oleandomycin, or spiramycin; penicillins, such as penicillin G and V, phenethicillin, methicillin, oxacillin, cloxacillin, dicloxacillin, floxacillin, nafcillin, ampicillin, amoxicillin, or carbenicillin; suflonamides; tetracyclines, such as tetracycline, oxytetracycline, chlortetracycline, methacycline, demeclocycline, rolitetracycline, doxycycline, or minocycline; trimethoprim-sulfamethoxazole; polypeptides, such as bacitracin, polymyxins, tyrothricin, or vancomycin; and miscellaneous antibiotics, such as lincomycin, clindamycin, or spectinomycin, in addition to oxytetracycline hydrochloride (OTC).

There are a plurality of germicides which may at least partially form the medical agent of the present invention, including phenols; cresols; resorcinols; substituted phenols; aldehydes; benzoic acid; salicyclic acid; iodine; iodophors, such as betadine; chlorophors, such as hypochlorites; peroxides; such as hydrogen peroxide and zinc peroxide; heavy metals and their salts, such as merbromin, silver nitrate, zinc sulfate; surface-active agents, such as benzalkonium chloride; furan derivatives, such as nitrofurazone; sulfur and thiosulfates; salicylanilides; and carbanilides.

The amount of the antibiotic or germicide present in an application of a marking varies with the nature of antibiotics or germicides employed and to some extent the method applying the marking as understood by one of ordinary skill in the art.

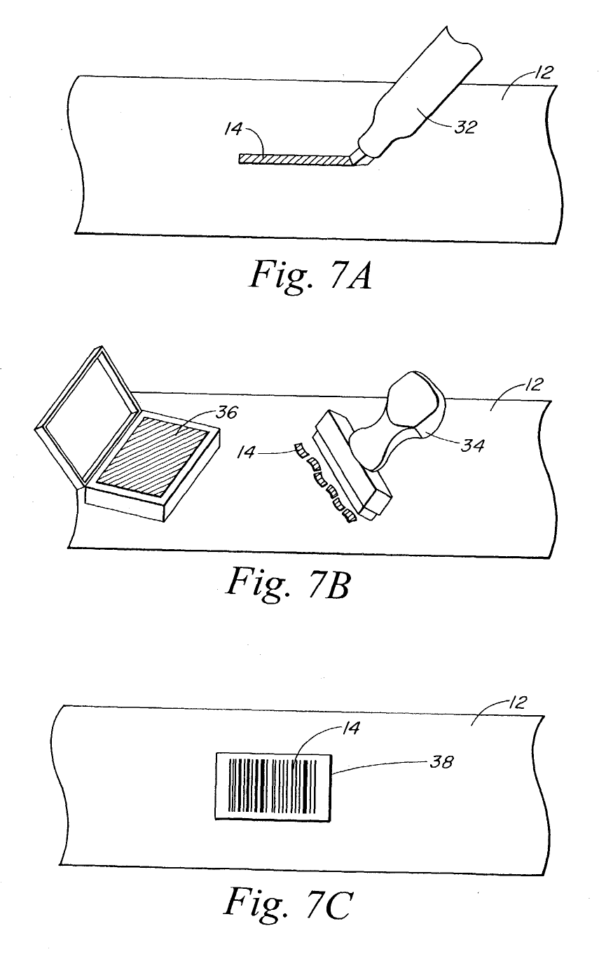

FIG. 7A illustrates one example device for applying the marking 14 to the tissue. A marker pen 32 containing the ink can be used to apply the marking 14 to the tissue 12. The clinical user draws the desired marking 14 directly on the tissue 12 with the ink containing one or more therapeutic agents. Different color marker pens 32 can contain different medication classifications or types of medication based on different color schemes. The marker pen 32 can also be utilized in forming simple color patterns, symbols, or text.

FIG. 7B illustrates a stamp device 34 embodiment. The stamp device 34 applies the marking 14 to the tissue 12. The stamp device 34 can obtain ink from an ink pad 36 that contains the desired ink with one or more agent. The stamp device 34 is then pressed against the tissue 12 to form the marking 14. A different color ink pad can contain a different medication classification or type of medication based on different color schemes. Another application can utilize thermal transfer from a secondary film loaded with transferable medicated ink.

FIG. 7C illustrates a tattoo in the form of a decal 38 applied to the tissue 12. The decal has the ink imbedded therein, with at least one agent included in the ink. The decal 38 illustrated is in the shape of a bar code that can be read by an infrared scanner to convey information concerning the agent or agents contained within the decal 38 and thus applied to the tissue 12. One of ordinary skill in the art will appreciate that the decal 38 can include codes or symbols other than a bar code. Some of the codes or symbols can be read by a user without the aid of a viewing device, and other codes or symbols require a form of machine vision, or other, technology to view interpret the information conveyed by the symbol or code. Alternative tattoo depositions of the ink can make use of a needle for direct ink injection into the tissue to form the marking 14.

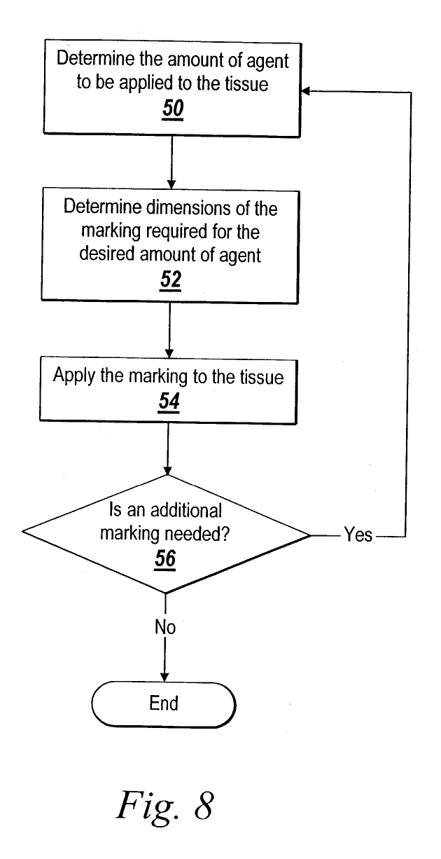

FIG. 8 illustrates an example method of determining an amount of agent to be applied to the tissue 12 of a patient in accordance with an illustrative embodiment of the present invention. First, a user determines the amount of agent desired to be applied to a the tissue 12 (step 50). Then the user determines dimensions of the marking 14 to be applied to deliver the desired amount of agent (step 52). The user then applies the marking 14 to the tissue 12 in accordance with the dimensions calculated for the desired agent dosage (step 54). After applying the marking 14, the user then determines whether additional agents or markings are necessary for the desired effect on the tissue 12 of the patient (step 56). If no additional markings 12 are required, the process is complete. If additional markings are required, the user can repeat the process. The user can apply different agents to the same area of tissue 12 as needed or apply more of the same agents with subsequent marker applications to increase dosage. The present invention can provide multiple ink markings 14 with different therapeutic effects and independent activation and/or release rates on the tissue 12 of the patient.

The markings 14 of the present invention enable the distribution of agents to a targeted location on a patient's body. The ink is relatively thin and unobtrusive to the applied surface. The marking 14 can further provide relevant information concerning the agents combined with the ink, as well as other characteristics of the ink and/or the agent, such as drug type, drug brand, drug dosage, dimensions, sizing, placement, orientation, and the like.

The present invention has many different therapeutic uses. More specifically, one clinical use for the marking 14 containing at least one agent is for application onto tissue 12. The tissue 12 can include both internal and external sides of a patient's skin, as well as any other tissue within the patient. In some instances, the tissue may only be accessible during a surgical or other medical procedure.

All identifiable and/or detectable drug exuding inks that form the markings 14 can be made as a permanent marking or as a temporary marking, which can be absorbed by the local tissue 12. More specifically, the marking 14 can have a relatively short term therapeutic effect, or the marking 14 can have a longer term, more permanent effect. A tattoo, for example, is representative of an ink that is a longer term application. Whereas, an ink that is applied and is absorbed in a matter of minutes or days has a shorter term therapeutic effect. Inks and agents combined with inks can have therapeutic effects ranging between the shorter term and longer term applications.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. It is intended that the present invention be limited only to the extent required by the appended claims and the applicable rules of law.

1. A therapeutic ink for forming a therapeutic marking on tissue of a patient, the therapeutic ink comprising:
a component providing an indication of location of the ink when applied to the tissue; and
at least one therapeutic agent component for creating a therapeutic effect when the therapeutic ink is applied to the tissue.

2. The therapeutic ink of claim 1, wherein the marking is applied with a marker.

3. The therapeutic ink of claim 1, wherein a dosage of the medical agent in the therapeutic ink can be determined by inspection of the marking.

4. The therapeutic ink of claim 1, wherein a dosage of the at least one therapeutic agent in the therapeutic ink is controlled by detectable dimensions of the marking on the tissue.

5. The therapeutic ink of claim 1, wherein a dosage of the at least one therapeutic agent in the therapeutic ink is determinable by visual detection of the marking.

6. The therapeutic ink of claim 1, wherein the at least one therapeutic ink comprises two or more therapeutic inks forming the marking and wherein the marking comprises a first color and a second color that differs from the first color.

7. The therapeutic ink of claim 1, wherein the marking comprises more than one type of therapeutic agent.

8. The therapeutic ink of claim 1, wherein the therapeutic agent comprises a diagnostic medical agent.

9. The therapeutic ink of claim 1, wherein the marking is permanently applied to the tissue.

10. The therapeutic ink of claim 1, wherein the marking is temporarily applied to the tissue.

11. The therapeutic ink of claim 1, wherein the therapeutic agent comprises an antioxidant agent.

12. The therapeutic ink of claim 1, wherein the therapeutic agent comprises an antihypertensive agent.

13. The therapeutic ink of claim 1, wherein the therapeutic agent comprises an anti-inflammatory agent.

14. The therapeutic ink of claim 1, wherein the therapeutic agent comprises a growth factor antagonist.

15. The therapeutic ink of claim 1, wherein the therapeutic agent comprises an antiplatelet agent.

16. The therapeutic ink of claim 1, wherein the therapeutic agent comprises an anticoagulant agent.

17. The therapeutic ink of claim 1, wherein the therapeutic agent comprises a thrombolytic agent.

18. The therapeutic ink of claim 1, wherein the therapeutic agent comprises drugs to alter lipid metabolism.

19. The therapeutic ink of claim 1, wherein the therapeutic agent comprises an ACE inhibitor.

20. The therapeutic ink of claim 1, wherein the therapeutic agent comprises at least one of an antiproliferative and an antineoplastic.

21. The therapeutic ink of claim 1, wherein the therapeutic agent comprises a tissue growth stimulant.

22. The therapeutic ink of claim 1, wherein the therapeutic agent comprises a chemical donor of at least one of Nitric oxide and Super Oxygenated 02.

23. The therapeutic ink of claim 1, wherein the therapeutic agent comprises promotion of hollow organ occlusion or thrombosis agents.

24. The therapeutic ink of claim 1, wherein the therapeutic agent comprises a functional protein and factor delivery agent.

25. The therapeutic ink of claim 1, wherein the therapeutic agent comprises a second messenger targeting agent.

26. The therapeutic ink of claim 1, wherein the therapeutic agent comprises an angiogenic agent.

27. The therapeutic ink of claim 1, wherein the therapeutic agent comprises an anti-angiogenic agent.

28. The therapeutic ink of claim 1, wherein the therapeutic agent comprises an inhibition of protein synthesis agent.

29. The therapeutic ink of claim 1, wherein the therapeutic agent comprises an anti-infective agent.

30. The therapeutic ink of claim 1, wherein the therapeutic agent comprises a gene delivery agent.

31. The therapeutic ink of claim 1, wherein the therapeutic agent comprises at least one of a tissue perfusion enhancer and a tissue absorption enhancer.

32. The therapeutic ink of claim 1, wherein the therapeutic agent comprises a nitric oxide donative derivative.

33. The therapeutic ink of claim 1, wherein the therapeutic agent comprises drug carrying nano-particles.

34. The therapeutic ink of claim 1, wherein the therapeutic agent comprises drug carrying micro-spheres.

35. The therapeutic ink of claim 1, wherein the therapeutic agent comprises at least one of an imaging agent and a contrast agent.

36. The therapeutic ink of claim 1, wherein the therapeutic agent comprises an anesthetic agent.

37. The therapeutic ink of claim 1, wherein the therapeutic agent comprises a descaling agent.

38. The therapeutic ink of claim 1, wherein the therapeutic agent comprises a cheomotherapeutic agent.

39. The therapeutic ink of claim 1, wherein the therapeutic agent is suitable for release into the body of the patient.

40. The therapeutic ink of claim 1, wherein the therapeutic agent is suitable for release into the tissue of the patient.

41. The therapeutic ink of claim 1, wherein the therapeutic agent includes an immobilized drug.

42. The therapeutic ink of claim 1, wherein the therapeutic agent exudes from the marking.

43. The therapeutic ink of claim 1, wherein the therapeutic agent is enclosed in liposomes.

44. The therapeutic ink of claim 1, wherein the ink marking is visible to the human eye without visual aid.

45. The therapeutic ink of claim 1, wherein the ink marking is visible to the human eye with use of a visual aid.

46. The therapeutic ink of claim 1, wherein the manner in which the ink marking is applied comprises at least one of a color code format, a shape format, a symbol format, and a size format to convey information.

47. The therapeutic ink of claim 1, wherein the manner in which the ink marking is applied comprises application of the ink marking to indicate at least one of drug type, drug brand, drug dosage, dimensions, sizing, placement, orientation, and trimming guidelines.

48. A method of applying a detectable therapeutic information conveying marking to tissue of a patient, comprising the steps of:
providing an applicator holding detectable therapeutic ink; and
applying a first marking of the detectable therapeutic ink to the tissue to apply a specific dosage of at least one therapeutic agent, wherein the dosage is controlled by the quantity of the first marking.

49. The method of claim 48, further comprising pre-treating the tissue prior to applying the first marking.

50. The method of claim 48, further comprising applying a second marking detectably different from the first marking.

51. The method of claim 48, wherein applying the first marking comprises applying multiple therapeutic agents to the tissue.

52. The method of claim 48, wherein at least the first marking is applied to the tissue by a marker pen.

53. The method of claim 48, wherein at least the first marking is applied to the tissue by a stamp device.

54. The method of claim 48, wherein at least the first marking is applied to the tissue in the form of a tattoo.

55. The method of claim 48, wherein the first marking comprises a first color.

56. The method of claim 55, further comprising a second marking formed of a second color that is visually differentiable from the first color.

57. The method of claim 48, wherein the therapeutic ink is suitable for release into the body of the patient.

58. The method of claim 48, wherein the therapeutic ink is suitable for release into the tissue of the patient.

59. A method of applying a therapeutic agent to tissue of a patient, comprising the steps of:
determining a length of detectable information conveying marking to be applied to the tissue according to the amount of medical agent desired; and
applying the determined length of marking to the tissue.

60. The method of claim 59, further comprising pre-treating the tissue prior to applying the marking.

61. The method of claim 59, further comprising applying a different marking that is detectably different from the marking.

62. The method of claim 59, wherein applying the determined length of marking comprises applying multiple drug medications to the tissue.

63. The method of claim 59, wherein the marking is applied to the tissue by a marker pen.

64. The method of claim 59, wherein the marking is applied to the tissue by a stamp device.

65. The method of claim 59, wherein the marking is applied to the tissue using thermal transfer.

66. The method of claim 59, wherein the marking is applied to the tissue using gas vapor deposition.

67. The method of claim 59, wherein the marking comprises a first color.

68. The method of claim 67, further comprising a different marking formed of a second color that is visually differentiable from the first color.

69. The method of claim 59, wherein the marking is suitable for release into a body of a patient.

70. The method of claim 59, wherein the medical ink is suitable for release into tissue of a patient.
